(19) United States
(12) Patent Application Publication
(10) Pub. No.: US 2006/0100353 A1
(43) Pub. Date: May 11, 2006

(54) Coating compositions for basecoats containing acrylic branched polymers

(76) Inventors: Robert John Barsotti, Franklinville, NJ (US); Christopher Scopazzi, Wilmington, DE (US); Frank T. Sobonya JR., Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/984,647

(22) Filed: Nov. 8, 2004

(51) Int. Cl. A61K 9/16

(52) U.S. Cl. 524/543

(57) ABSTRACT

This invention relates to rapid drying coating compositions that are particularly useful for automotive and truck refinish applications; the coating composition is pigmented and contains a branched acrylic polymer and is particularly useful as a basecoat for a basecoat clear coat finish; the coating composition may preferably be used as a lacquer coating, which dries via solvent evaporation absent any substantial crosslinking occurring or it optionally may contain a polyisocyanate crosslinking agent and be used a clear topcoat.

### FIELD OF THE INVENTION

This invention relates to rapid drying coating compositions that are particularly useful for automotive refinish and for automotive OEM (Original Equipment Manufacture) applications.

### BACKGROUND OF THE INVENTION

The typical finish on an automobile or truck body comprises an electrodeposited primer layer, an optional primer or primer surfacer layer over the electrodeposited layer and then a pigmented base coat layer and over the pigmented base coat layer, a clear coat layer is applied. A pigmented mono-coat may be used in place of the base coat/clear coat. A number of clear and pigmented lacquers have been utilized as automotive OEM and automotive refinish coatings, such as, primers, basecoats and clear coats. A combination of rapid drying times and outstanding physical properties, such as, chip and humidity resistance, excellent adhesion and good DOI (distinctness of image) are very desirable characteristic that these compositions should have.

In refinishing automobiles and trucks, the damaged painted areas having dents, mars, scratches and the like are sanded or ground out by mechanical means in and around the damaged area. Sometimes, the original coating is stripped off from a portion or off the entire auto or truck body to expose the substrate (e.g., bare metal or plastic composite) underneath. After repairing the damage, the repaired surface is coated and the applied layers are dried and cured.

A key concern to a refinish customer is that the color match of the repair finish matches the original finish and that the applied coating has excellent physical properties, such as chip and humidity resistance, and good adhesion and DOI.

Another key concern of the automobile and truck refinish industry is productivity, i.e., the ability to complete an entire refinish operation in the least amount of time. To accomplish a high level of productivity, any coatings applied need to have the ability to dry at ambient or elevated temperature conditions in a relatively short period of time. The term “dry” means that the resulting finish is physically dry to the touch in a relatively short period of time. This minimizes dirt pick-up and allows for movement of the vehicle to another location, and, in the case of the basecoat, allows for the application of the subsequent clear coat.

Current commercially available pigmented base coat or mono coat refinish coating compositions do not have these unique characteristics of rapidly drying under ambient temperature conditions along with the ability to form a finish having improved chip and humidity resistance and good adhesion and DOI. It would be advantageous to have a refinish coating composition lacquer with this unique combination of properties.

### STATEMENT OF THE INVENTION

A coating composition comprising 10% to 95% by weight, based on the weight of the coating composition of a liquid organic carrier, 5% to 90% by weight, based on the weight of the coating composition of a binder and optionally, pigment in a pigment to binder weight ratio of 0.1/100 to 200/100;
wherein the binder comprises a branched acrylic polymer having a glass transition temperature of −10° C. to 100° C. and a weight average molecular weight of 8000 to 150,000 comprising macromonomers formed from free radical polymerized ethylenically unsaturated monomers substantially having a terminal ethylenically unsaturated group polymerized with ethylenically unsaturated monomers, thereby forming a branched acrylic polymer having a backbone of polymerized ethylenically unsaturated monomers and macromonomer branch chains;
wherein the ethylenically unsaturated monomers comprise a mixture of at least two different ethylenically unsaturated monomers wherein at least one of the monomers has the formula
   CH₂═CXY
where X is H or CH₃ and
Y contains a group selected from the group of carboxyl, hydroxyl, primary amine, secondary amine, or tertiary amine.

This coating composition may preferably be used as a lacquer coating, which dries via solvent evaporation absent any substantial crosslinking occurring or it optionally may contain a polyisocyanate crosslinking agent.

### DETAILED DESCRIPTION OF THE PREFFERED EMBODIMENTS

The features and advantages of the present invention will be more readily understood, by those of ordinary skill in the art, from reading the following detailed description. It is to be appreciated those certain features of the invention, which are, for clarity, described above and below in the context of separate embodiments, may also be provided in combination in a single embodiment. Conversely, various features of the invention that are, for brevity, described in the context of a single embodiment, may also be provided separately or in any sub-combination. In addition, references in the singular may also include the plural (for example, “a” and “an” may refer to one, or one or more) unless the context specifically states otherwise.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. Also, the disclosure of these ranges is intended as a continuous range including every value between the minimum and maximum values.

All patents, patent applications and publications referred to herein are incorporated by reference in their entirety.

As used herein:

“Number average molecular weight” and “weight average molecular weight” are determined by gel permeation chromatography (GPC) using a high performance liquid chromatograph (HPLC) supplied by Hewlett-Packard, Palo Alto, Calif. Unless stated otherwise, the liquid phase used was tetrahydrofuran and the standard used was polymethyl methacrylate.

“Polymer solids” or “Binder solids” means a polymer or binder in its dry state.

“Acrylic polymer” means polymerized “(meth)acrylates” which mean acrylates and methacrylates.

“Tg” (glass transition temperature) of a polymer is a measure of the hardness of the polymer. The higher the Tg, the harder the coating. Tg is described in Principles of Polymer Chemistry (1953), Cornell University Press. The Tg can be actually measured or it can be calculated as described by Fox in Bull. Amer. Physics Soc., 1, 3, page 123 (1956). Tg, as used herein, refers to the actually measured values. For measurement of the Tg of a polymer, differential scanning calorimetry (DSC) was used.

“Lacquer” is a coating composition, which dries via solvent evaporation without any substantial crosslinking of the binder of the coating composition.

The coating composition of this invention contains approximately 10% to 95% by weight, based on the weight of the coating composition, of a liquid organic carrier and 5% to 90% by weight, based on the weight of the coating composition, of a binder of a branched acrylic polymer and optionally contains pigments in a pigment to binder weight ratio of 0.1/100 to 200/100.

### Branch Acrylic Polymer

The branched acrylic polymer used in the coating composition of the present invention has a weight average molecular weight ranging from 8000 to 150,000, alternately, from 10,000 to 100,000 and still further alternately, 15,000 to 80,000, Tg ranging from −10° C. to +100° C., alternately, from 0° C. to 80° C., and further alternately, from 10° C. to 75° C.

The branched acrylic polymer can be described as having a backbone of polymerized ethylenically unsaturated monomer and macromonomer branch chains. The macromonomers are formed from free radically polymerized ethylenically unsaturated monomers and substantially have a terminal ethylenically unsaturated group that is polymerized with the ethylenically unsaturated backbone monomers to form the branched acrylic polymer.

At least two different ethylenically unsaturated monomers are used to form the backbone and the macromonomers of the branched acrylic polymer. At least one of these ethylenically unsaturated monomers have the formula
CH₂═CXY
where X is H or CH₃ and Y contains a group selected from carboxyl, hydroxyl, primary amine, secondary amine, or tertiary amine.

Preferably, the branched acrylic polymer contains about 30-70% by weight of the backbone and 70-30% by weight of substantially linear branch chains. This branched acrylic polymer may be in solution, soluble in the carrier solvent, or the solvent may be stripped during the synthesis and replaced by a non-solvent, such as aliphatic hydrocarbons, to form a dispersed polymer referred to as a solvent responsive dispersion (SRD).

These macromonomers which form the branch chains of the polymer comprises polymerized ethylenically unsaturated monomers and substantially have one terminal ethylenically unsaturated moiety and have a weight average molecular weight (MW) of 500-40,000, alternately, 1,000 to 25,000. About 15-85% (by weight), and alternately, 30-70% (by weight), of the macromonomers are copolymerized with 85-15%, alternately, 70-30% of a blend of other ethylenically unsaturated monomers, which form the backbone of the branch acrylic polymer. At least 2%, alternately, 2-40% by weight, of the monomers have functional groups in the branches or the backbone or in both that are capable of reacting with a crosslinking agent, such as a polyisocyanate, if present in the coating composition.

The branched acrylic polymer may be prepared by polymerizing ethylenically unsaturated monomers that comprise the backbone in the presence of macromonomers, each macromonomer having at least one ethylenic unsaturation component. The acrylic polymer can be described as having a backbone having a plurality of macromonomer chains attached thereto.

It is to be understood that the backbone or macromonomers referred to as having functionality may be part of a mixture of macromonomers of which a portion do not have any functionality or variable amounts of functionality. It is also understood that, in preparing any backbone or macromonomers, there is a normal distribution of functionality.

Macromonomers can be prepared by conventional techniques as shown in Hazan et al U.S. Pat. No. 5,066,698 issued Nov. 19, 1991 (see Example 1) using conventional catalysts.

In an alternative method, a catalytic chain transfer agent is used to ensure that the resulting macromonomer only has one terminal ethylenically unsaturated group which will polymerize with the backbone monomers to form the branched acrylic polymer. Typically, in the first step of the process for preparing the macromonomer, the monomers are blended with an inert organic solvent and a cobalt chain transfer agent and heated usually to the reflux temperature of the reaction mixture. In subsequent steps, additional monomers and cobalt catalyst and conventional polymerization catalyst are added and polymerization is continued until a macromonomer is formed of the desired molecular weight.

Preferred cobalt chain transfer agents or catalysts are described in U.S. Pat. No. 4,680,352 to Janowicz et al and U.S. Pat. No. 4,722,984 to Janowicz. Alternate cobalt II (Co⁺²) are pentacyanocobaltate (II), diaquabis(borondifluorodimethyl-glyoximato) cobaltate(II) and diaquabis(borondifluorophenylglyoximato) cobaltate (II). Cobalt (III) (Co⁺³) versions of these catalysts are also alternate catalysts. Typically these chain transfer agents are used at concentrations of about 5-1000 ppm based on the monomers used.

The macromonomer is preferably formed in a solvent or solvent blend using a free radical initiator and a Co (II) or Co (III) chelate chain transfer agent.

Examples of solvents are aromatics, aliphatics, ketones, glycol ethers, acetates, alcohols as, e.g., methyl ethyl ketone, isopropyl alcohol, n-butyl glycol ether, n-butyl diethylene glycol ether, propylene glycol methyl ether acetate, propylene glycol methyl ether, and n-butanol.

Peroxy- and azo-initiators (0.1-5% weight on monomer) can be used in the synthesis of the macromonomers (provided that these initiators do not poison the activity of the cobalt chain transfer agent) in the presence of 2-5,000 ppm (on total monomer) or Co (II) chelate in the temperature range between 70-160° C., alternately, azo-type initiators as, e.g., 2,2′-azobis (2,4 dimethylpentane nitrile), 2,2′-azobis (2-methylpropane nitrile), 2,2′-azobis (2-methylbutane nitrile), 1,1′-azo (cyclohexane carbonitrile) and 4,4′-azobis (4-cyanopentanoic) acid can be used.

After the macromonomer is formed as described above, solvent is optionally stripped off and the backbone monomers are added to the macromonomer along with additional solvent and polymerization initiators. Any of the aforementioned azo-type initiators can be used as can other suitable initiators, such as peroxides and hydroperoxides. Typical of such initiators are di-tertiarybutyl peroxide, dicumylperoxide, tertiaryamyl peroxide, cumenehydroperoxide, di(n-propyl) peroxydicarbonate, peresters such as amyl peroxyacetate and the like. Commercially available peroxy type initiators include, e.g., t-butylperoxide or Triganox®. B from AKZO, t-butylperacetate or Triganox®) FC50 from AKZO, t-butylperbenzoate or Triganox® C from AKZO, and t-butylperpivalate or Triganox® 25 C-75 from AKZO.

Polymerization is continued at or below the reflux temperature of the reaction mixture until the branched acrylic polymer is formed of the desired molecular weight.

During the polymerization or afterward, non-solvent(s) for the backbone, such as aliphatic hydrocarbons, may be added to form low viscosity sprayable polymer dispersion rather than a polymer solution having a relatively high viscosity which would require further dilution with solvents for spraying thereby increasing the VOC content of the composition.

Typical solvents that can be used to form the macromonomer or the branched acrylic polymer are ketones, such as methyl ethyl ketone, isobutyl ketone, ethyl amyl ketone, acetone, alcohols, such as methanol, ethanol, isopropanol, esters, such as ethyl acetate, glycols, such as ethylene glycol, propylene glycol, ethers, such as tetrahydrofuran, ethylene glycol mono butyl ether and the like.

Some of the typical monomers that are used to form the branched acrylic polymer have the formula CH₂═CXY where X is H or CH₃ and Y contains groups that are either carboxyl, hydroxyl, primary amine, secondary amine, or tertiary amine.

Ethylenically unsaturated monomers containing hydroxy functionality include hydroxy alkyl acrylates and hydroxy alkyl methacrylates, wherein the alkyl group has 1 to 4 carbon atoms can be used. Suitable monomers include 2-hydroxy ethyl acrylate, 2-hydroxy ethyl methacrylate, 2-hydroxy propyl acrylate, 2-hydroxy propyl methacrylate, 2-hydroxy isopropyl acrylate, 2-hydroxy isopropyl methacrylate, 2-hydroxy butyl acrylate, 2-hydroxy butyl methacrylate, and the like, and mixtures thereof. Hydroxy functionality may also be obtained from monomer precursors, for example, the epoxy group of a glycidyl methacrylate unit in a polymer. Such an epoxy group may be converted, in a post polymerization reaction with water or a small amount of acid, to a hydroxy group.

Typical polymerizable carboxyl functional monomers that can be use are acrylic acid, methacrylic acid, maleic acid, itaconic acid, maleic, itaconic and fumaric anhydride and their half esters. Methacrylic and acrylic acid are preferred. Other acid functional monomers that can be used are ethylenically unsaturated sulfonic, sulfinic, phosphoric or phosphonic acid and esters thereof; typically, styrene sulfonic acid, acrylamido methyl propane sulfonic acid, vinyl phosphonic or phosphoric acid and its esters.

Typically useful amine functional monomers are aminoalkyl (meth)acrylates, such as tertiarybutylaminoethyl (meth)acrylate, N-methylaminoethyl (meth)acrylate and diethylaminoethyl (meth)acrylate.

Other typical monomers that can be used to form the backbone or the macromonomers are, for example, but not limited to, (meth)acrylic acid esters of straight-chain or branched monoalcohols of 1 to 20 carbon atoms. Preferred esters are alkyl (meth)acrylates having 1-12 carbons in the alkyl group, such as methyl acrylate, ethyl acrylate, propyl acrylate, isopropyl acrylate, butyl acrylate, pentyl acrylate, hexyl acrylate, 2-ethyl hexyl acrylate, nonyl acrylate, lauryl acrylate, methyl methacrylate, ethyl methacrylate, propyl methacrylate, isopropyl methacrylate, butyl methacrylate, pentyl methacrylate, hexyl methacrylate, 2-ethyl hexyl methacrylate, nonyl methacrylate, lauryl methacrylate and the like. Isobornyl methacrylate and isobornyl acrylate monomers can be used. Cycloaliphatic acrylates methacrylates can be used, such as trimethylcyclohexyl acrylate, t-butyl cyclohexyl acrylate, cyclohexyl methacrylate, isobornyl methacrylate, 2-ethylhexyl methacrylate, and the like. Aryl acrylates and methacrylates, such as benzyl acrylate and benzyl methacrylate also can be used.

Suitable other olefinically unsaturated comonomers that can be used include: acrylamide and methacrylamide and derivatives as alkoxy methyl (meth) acrylamide monomers, such as methacrylamide, and N-isobutoxymethyl methacrylamide; diesters; vinyl aromatics such as styrene, alpha methyl styrene and vinyl toluene; and polyethylene glycol monoacrylates and monomethacrylates.

Optionally, the macromonomer branches or the backbone or both of the branched acrylic polymer can contain at least 2% and up to 40% by weight, based on the weight of the branched acrylic polymer, of polymerized ethylenically unsaturated monomers containing functional groups which will react with a crosslinking agent, such as a polyisocyanate crosslinking agent in the event such a crosslinking agent is present in the coating composition.

Particularly useful branched acrylic polymers include the following:

a branched acrylic polymer having a backbone of polymerized (meth)acrylate monomers, styrene monomers, (meth)acrylic acid monomers, and hydroxy-functional (meth)acrylate and branches of polymerized macromonomers having a weight average molecular weight of about 500-20,000 and containing polymerized alkyl (meth)acrylate monomers, isobornyl (meth)acrylate monomers and hydroxy alkyl (meth)acrylate monomers. One particularly useful polymer comprises a backbone of polymerized methyl methacrylate, hydroxy ethyl acrylate, acrylic acid, methyl acrylate and styrene and the macromonomer chain comprise polymerized ethyl hexyl methacrylate, isobornyl methacrylate, butyl methacrylate and hydroxy ethyl acrylate.

### Pigments

The novel composition can be pigmented to form a colored mono coat, basecoat, primer or primer surfacer. Generally, pigments are used in a pigment to binder weight ratio (P/B) of 0.1/100 to 200/100; preferably, for base coats in a P/B of 1/100 to 50/100. If used as primer or primer surfacer higher levels of pigment are used, e.g., 50/100 to 200/100. The pigments can be added using conventional techniques, such as sand-grinding, ball milling, attritor grinding, two roll milling to disperse the pigments. The mill base is blended with the film-forming constituents. This composition can be applied and cured as described below. The pigment component of this invention may be any of the generally well-known pigments or mixtures thereof used in coating formulations, as reported, e.g., in Pigment Handbook, T. C. Patton, Ed., Wiley-lnterscience, New York, 1973.

Any of the conventional pigments used in coating compositions can be utilized in the composition such as the following: metallic oxides, metal hydroxide, metal flakes, chromates, such as lead chromate, sulfides, sulfates, carbonates, carbon black, silica, talc, china clay, phthalocyanine blues and greens, organo reds, organo maroons, pearlescent pigments and other organic pigments and dyes. If desired, chromate-free pigments, such as barium metaborate, zinc phosphate, aluminum triphosphate and mixtures thereof, can also be used.

Suitable flake pigments include bright aluminum flake, extremely fine aluminum flake, medium particle size aluminum flake, and bright medium coarse aluminum flake; mica flake coated with titanium dioxide pigment also known as pearl pigments. Suitable colored pigments include titanium dioxide, zinc oxide, iron oxide, carbon black, mono azo red toner, red iron oxide, quinacridone maroon, transparent red oxide, dioxazine carbazole violet, iron blue, indanthrone blue, chrome titanate, titanium yellow, mono azo permanent orange, ferrite yellow, mono azo benzimidazolone yellow, transparent yellow oxide, isoindoline yellow, tetrachloroisoindoline yellow, anthanthrone orange, lead chromate yellow, phthalocyanine green, quinacridone red, perylene maroon, quinacridone violet, pre-darkened chrome yellow, thio-indigo red, transparent red oxide chip, molybdate orange, and molybdate orange red.

### Crosslinking Component

If the novel composition is used as a clear coating composition, a crosslinking component generally is required to provide the level of durability and weatherability required for automotive and truck topcoats. Typically, polyisocyanates are used as the crosslinking agents. Suitable polyisocyanate has on average 2 to 10, alternately 2.5 to 8 and further alternately 3 to 8 isocyanate functionalities. Typically the coating composition has a ratio of isocyanate groups on the polyisocyanate in the crosslinking component to crosslinkable groups of the branched acrylic polymer ranges from 0.25/1 to 3/1, alternately from 0.8/1 to 2/1, further alternately from 1/1 to 1.8/1.

Examples of suitable polyisocyanates include aromatic, aliphatic or cycloaliphatic di-, tri- or tetra-isocyanates, including polyisocyanates having isocyanurate structural units, such as, the isocyanurate of hexamethylene diisocyanate and isocyanurate of isophorone diisocyanate; the adduct of 2 molecules of a diisocyanate, such as, hexamethylene diisocyanate; uretidiones of hexamethylene diisocyanate; uretidiones of isophorone diisocyanate or isophorone diisocyanate; isocyanurate of meta-tetramethylxylylene diisocyanate; and a diol such as, ethylene glycol.

Additional examples of suitable polyisocyanates include 1,2-propylene diisocyanate, trimethylene diisocyanate, tetramethylene diisocyanate, 2,3-butylene diisocyanate, hexamethylene diisocyanate, octamethylene diisocyanate, 2,2,4-trimethyl hexamethylene diisocyanate, 2,4,4-trimethyl hexamethylene diisocyanate, dodecamethylene diisocyanate, omega, omega-dipropyl ether diisocyanate, 1,3-cyclopentane diisocyanate, 1,2-cyclohexane diisocyanate, 1,4-cyclohexane diisocyanate, isophorone diisocyanate, 4-methyl-1,3-diisocyanatocyclohexane, trans-vinylidene diisocyanate, dicyclohexylmethane-4,4′-diisocyanate, 3,3′-dimethyl-dicyclohexylmethane4,4′-diisocyanate, a toluene diisocyanate, 1,3-bis(1-isocyanato1-methylethyl)benzene, 1,4-bis(1-isocyanato-1-methylethyl)benzene, 1,3-bis(isocyanatomethyl)benzene, xylene diisocyanate, 1,5-dimethyl-2,4-bis(isocyanatomethyl)benzene, 1,5-dimethyl-2,4-bis(2-isocyanatoethyl)benzene, 1,3,5-triethyl-2,4-bis(isocyanatomethyl)benzene, 4,4′-diisocyanatodiphenyl, 3,3′-dichloro-4,4′-diisocyanatodiphenyl, 3,3′-diphenyl-4,4′-diisocyanatodiphenyl, 3,3′-dimethoxy-4,4′-diisocyanatodiphenyl, 4,4′-diisocyanatodiphenylmethane, 3,3′-dimethyl-4,4′-diisocyanatodiphenyl methane, and diisocyanatonaphthalene.

Polyisocyanates having isocyanaurate structural units can be used, for example, the adduct of 2 molecules of a diisocyanate, such as, hexamethylene diisocyanate or isophorone diisocyanate, and a diol such as ethylene glycol; the adduct of 3 molecules of hexamethylene diisocyanate and 1 molecule of water (available under the trademark Desmodur® N from Bayer Corporation of Pittsburgh, Pa.); the adduct of 1 molecule of trimethylol propane and 3 molecules of toluene diisocyanate (available under the trademark Desmodur® L from Bayer Corporation ); the adduct of 1 molecule of trimethylol propane and 3 molecules of isophorone diisocyanate or compounds, such as 1,3,5-triisocyanato benzene and 2,4,6-triisocyanatotoluene; and the adduct of 1 molecule of pentaerythritol and 4 molecules of toluene diisocyanate.

The coating composition containing a crosslinking component preferably includes one or more catalysts to enhance crosslinking of the components on curing. Generally, the coating composition includes in the range of from 0.001 percent to 5 percent, alternately in the range of from 0.005 to 2 percent, further alternately in the range of from 0.01 percent to 2 percent and still further alternately in the range of from 0.01 percent to 1.2 percent of the catalyst, the percentages being in weight percentages based on the total weight of the binder.

Suitable catalysts for polyisocyanate can include one or more tin compounds, tertiary amines or a combination thereof. Suitable tin compounds include dibutyl tin dilaurate, dibutyl tin diacetate, stannous octoate, and dibutyl tin oxide. Dibutyl tin dilaurate is preferred. Suitable tertiary amines include triethylene diamine. One commercially available catalyst that can be used is Fastcat® 4202 dibutyl tin dilaurate sold by Elf-Atochem North America, Inc. Philadelphia, Pa. Carboxylic acids, such as acetic acid, may be used in conjunction with the above catalysts to improve the viscosity stability of two component coatings.

### Branched Copolyester Polyol

The novel coating composition of this invention optionally includes a branched copolyester polyol in the range of from 5 percent to 50 percent, alternately, in the range of from 10 percent to 40 percent, and further alternately in the range of from 15 percent to 30 percent; the percentages being in weight percentages based on the total weight of the binder.

These branched copolyesters polyols and the preparation thereof are described in WO 03/070843 published Aug. 28, 2003, which is hereby incorporated by reference.

The branched copolyester polyol has a number average molecular weight not exceeding 30,000, alternately in the range of from 1,000 to 30,000, further alternately in the range of 2,000 to 20,000, and still further alternately in the range of 5,000 to 15,000. The copolyester polyol has hydroxyl groups ranging from 5 to 200 per polymer chain, preferably 6 to 70, and more preferably 10 to 50, and carboxyl groups ranging from 0 to 40 per chain, preferably 1 to 40, more preferably 1 to 20 and most preferably 1 to 10. The Tg (glass transition temperature) of the copolyester polyol ranges from −70° C. to 50° C., preferably from −65° C. to 40° C., and more preferably from −60° C. to 30° C.

The branched copolyester polyol is conventionally polymerized from a monomer mixture containing a chain extender selected from the group consisting of a hydroxy carboxylic acid, a lactone of a hydroxy carboxylic acid and a combination thereof; and one or more hyper branching monomers.

### Other Ingredients

The following additional ingredients can be included in the coating composition in amounts of 0.1% to 98% by weight and alternately in the range of 50% to 95% by weight, all based on the weight of the binder of the coating composition.

Typical additives include conventional polyesters, alkyd resins, acrylic alkyd resins, cellulose acetate butyrates, iminated acrylic polymers, ethylene vinyl acetate co-polymers, nitrocellulose, plasticizers or any combination thereof.

Useful acrylic alkyd polymers having a weight average molecular weight ranging from 3,000 to 100,000 and a Tg ranging from 0° C. to 100° C. are conventionally polymerized from a monomer mixture that can include one or more of the following monomers: an alkyl (meth)acrylate, for example, methyl (meth)acrylate, butyl (meth)acrylate, ethyl (meth)acrylate, 2-ethyl hexyl (meth)acrylate; a hydroxy alkyl (meth)acrylate, for example, hydroxy ethyl (meth)acrylate, hydroxy propyl (meth)acrylate, hydroxy butyl (meth)acrylate; (meth)acrylic acid; styrene; and alkyl amino alkyl (meth)acrylate, for example, diethylamino ethyl (meth)acrylate or t-butyl aminoethyl methacrylate; and one or more of the following drying oils: vinyl oxazoline drying oil esters of linseed oil fatty acids, tall oil fatty acids or tung oil fatty acids.

One preferred polymer is polymerized from a monomer mixture that contains an alkyl (meth)acrylate, hydroxy alkyl acrylate, alkylamino alkyl acrylate and vinyl oxazoline ester of drying oil fatty acids.

Suitable iminated acrylic polymers can be obtained by reacting acrylic polymers having carboxyl groups with an alkylene imine, such as propylene imine.

Typically useful polyesters have a weight average molecular weight ranging from 1000 to 30,000 and a Tg in the range of −50° C. to +100° C. Some of the other suitable polyesters are also listed in U.S. Pat. No. 6,221,494 on column 5 and 6, which is incorporated herein by reference. The suitable polyester is the esterification product of an aliphatic or aromatic dicarboxylic acid, a polyol, a diol, an aromatic or aliphatic cyclic anhydride and a cyclic alcohol. One preferred polyester is the esterification product of adipic acid, trimethylol propane, hexanediol, hexahydrophathalic anhydride and cyclohexane dimethylol.

Suitable cellulose acetate butyrates are supplied by Eastman Chemical Co., Kingsport, Tenn. under the trade names CAB-381-20 and CAB-531-1 and are preferably used in an amount of 0.1% to 20% by weight based on the weight of the binder.

A suitable ethylene-vinyl acetate co-polymer (wax) is supplied by Honeywell Specialty Chemicals—Wax and Additives, Morristown, N.J., under the trade name A-C® 405 (T) Ethylene—Vinyl Acetate Copolymer.

Suitable nitrocellulose resins preferably have a viscosity of about ½-6 seconds. Preferably, a blend of nitrocellulose resins is used. Optionally, the lacquer can contain ester gum and castor oil.

Suitable alkyd resins are the esterification products of a drying oil fatty acid, such as linseed oil and tall oil fatty acid, dehydrated castor oil, a polyhydric alcohol, a dicarboxylic acid and an aromatic monocarboxylic acid. Typical polyhydric alcohols that can be used to prepare the alkyd resin used in this invention are glycerine, pentaerythritol, trimethylol ethane, trimethylol propane; glycols, such as ethylene glycol, propylene glycol, butane diol and pentane diol. Typical dicarboxylic acids or anhydrides that can be used to prepare the alkyd resin are phthalic acid, phthalic anhydride, isophthalic acid, terephthalic acid maleic, and fumaric acid. Typical monocarboxylic aromatic acids are benzoic acid, paratertiary butylbenzoic acid, phenol acetic acid and triethyl benzoic acid. One preferred alkyd resin is a reaction product of an acrylic polymer and an alkyd resin.

Suitable plasticizers include butyl benzyl phthalate, dibutyl phthalate, triphenyl phosphate, 2-ethylhexylbenzyl phthalate, dicyclohexyl phthalate, diallyl toluene phthalate, dibenzyl phthalate, butylcyclohexyl phthalate, mixed benzoic acid and fatty oil acid esters of pentaerythritol, poly(propylene adipate) dibenzoate, diethylene glycol dibenzoate, tetrabutylthiodisuccinate, butyl phthalyl butyl glycolate, acetyltributyl citrate, dibenzyl sebacate, tricresyl phosphate, toluene ethyl sulfonamide, the di-2-ethyl hexyl ester of hexamethylene diphthalate, and di(methyl cyclohexyl) phthalate. One preferred plasticizer of this group is butyl benzyl phthalate.

If desired, the coating composition can include metallic driers, chelating agents, or a combination thereof. Suitable organometallic driers include cobalt naphthenate, copper naphthenate, lead tallate, calcium naphthenate, iron naphthenate, lithium naphthenate, lead naphthenate, nickel octoate, zirconium octoate, cobalt octoate, iron octoate, zinc octoate, and alkyl tin dilaurates, such as dibutyl tin dilaurate. Suitable chelating agents include aluminum monoisopropoxide monoversatate, aluminum (monoisopropyl)phthalate, aluminum diethoxyethoxide monoversatate, aluminum trisecondary butoxide, aluminum diisopropoxide monoacetacetic ester chelate and aluminum isopropoxide.

Also, polytrimethylene ether diols may be used as an additive having a number average molecular weight (Mn) in the range of from 500 to 5,000, alternately in the range of from 1,000 to 3,000; a polydispersity in the range of from 1.1 to 2.1 and a hydroxyl number in the range of from 20 to 200. The preferred polytrimethylene ether diol has a Tg of −75° C. Copolymers of polytrimethylene ether diols are also suitable. For example, such copolymers are prepared by copolymerizing 1,3-propanediol with another diol, such as, ethane diol, hexane diol, 2-methyl-1,3-propanediol, 2,2-dimethyl-1,3-propanediol, trimethylol propane and pentaerythritol, wherein at least 50% of the copolymer results from 1,3-propanediol. A blend of a high and low molecular weight polytrimethylene ether diol can be used wherein the high molecular weight diol has an Mn ranging from 1,000 to 4,000 and the low molecular weight diol has an Mn ranging from 150 to 500. The average Mn of the diol should be in the range of 1,000 to 4,000. It should be noted that, the polytrimethylene ether diols suitable for use in the present invention can include polytrimethylene ether triols and other higher functionality polytrimethylene ether polyols in an amount ranging from 1% to 20%, by weight, based on the weight of the polytrimethylene ether diol. It is believed that the presence of polytrimethylene ether diols in the crosslinked coating composition of this invention can improve the chip resistance of a coating resulting therefrom.

Additional details of the foregoing additives are provided in U.S. Pat. Nos. 3,585,160, 4,242,243, 4,692,481, and U.S. Pat. No. Re 31.309, which are incorporated therein by reference.

If the novel composition is to be used as a clear coat for the exterior of automobiles and trucks, 0.1 weight percent to 5 weight percent, alternately, 1 weight percent to 2.5 weight percent and further alternately, 1.5 weight percent to 2 weight percent, based on the weight of the total weight of the binder, of an ultraviolet light stabilizer or a combination of ultraviolet light stabilizers and absorbers can be added to the clear coating composition to improve weatherability. These stabilizers include ultraviolet light absorbers, screeners, quenchers and specific hindered amine light stabilizers. Also, 0.1 weight percent to 5 weight percent, based on the total weight of the binder of an antioxidant can be added. Most of the foregoing stabilizers are supplied by Ciba Specialty Chemicals, Tarrytown, N.Y.

The novel composition of this invention preferably is in the form of a dispersion wherein at least the branched acrylic polymer of the binder is dispersed in an organic liquid carrier. The solids level of the coating of the present invention can vary in the range of from 5 percent to 90 percent, alternately in the range of from 10 percent to 85 percent and further alternately in the range of from 15 percent to 70 percent, all percentages being based on the total weight of the coating composition.

To form a dispersion, the branched acrylic polymer is prepared using conventional organic solvents and then inverted into a dispersion by the addition of an organic non-solvent. A typical non-solvent that can be used is heptane and other such non-solvents that are known to those skilled in the art can be used. One method that can be used to form a polymeric organic dispersion is taught in Barsotti et al. U.S. Pat. No. 5,412,039, which is hereby incorporated by reference. The coating composition of the present invention can further contain at least one organic solvent typically selected from the group consisting of aromatic hydrocarbons, such as, petroleum naphtha or xylenes; ketones, such as, methyl amyl ketone, methyl isobutyl ketone, methyl ethyl ketone or acetone; esters, such as butyl acetate or hexyl acetate; and glycol ether esters, such as, propylene glycol monomethyl ether acetate. The amount of organic solvent added depends upon the desired solids level as well as the desired amount of VOC of the composition.

### Application

In use, a layer of the novel composition is typically applied to a substrate by conventional techniques, such as, spraying, electrostatic spraying, roller coating, dipping or brushing. Spraying and electrostatic spraying are preferred application methods. When used as a pigmented coating composition, e.g., as a basecoat or a pigmented top coat, the coating thickness can range from 10 to 85 micrometers, preferably from 12 to 50 micrometers and when used as a primer, the coating thickness can range from 10 to 200 micrometers, preferably from 12 to 100 micrometers. When used as a clear coating, the thickness is in the range of from 25 micrometers to 100 micrometers. The coating composition can be dried at ambient temperatures or can be dried upon application for about 2 to 60 minutes at elevated drying temperatures ranging from about 50° C. to 100° C.

In a typical clear coat/base coat application, a layer of conventional clear coating composition is applied over the basecoat of the novel composition of this invention by the above conventional techniques, such as, spraying or electrostatic spraying. Generally, a layer of the basecoat is flashed for 1 minute to two hours under ambient or elevated temperatures before the application of the clear coating composition or dried at elevated temperatures shown above. Suitable clear coating compositions can include two-pack isocyanate crosslinked compositions, such as 72200S ChromaPremier® Productive Clear blended with an activator, such as 12305S ChromaPremier®Activator, or 3480S Low VOC Clear composition activated with 194S Imron Select® Activator. Isocyanate free crosslinked clear coating compositions, such as 1780S Iso-Free Clearcoat activated with 1782S Converter and blended with 1775S Mid-Temp Reducer are also suitable. Suitable clear lacquers can include 480S Low VOC Ready to Spray Clear composition. All the forgoing clear coating compositions are supplied by DuPont (E.I. Dupont de Nemours and Company, Wilmington, Del.).

If the coating composition of the present invention contains a crosslinking agent, such as a polyisocyanate, the coating composition can be supplied in the form of a two-pack coating composition in which the first-pack includes the branched acrylic polymer and the second pack includes the crosslinking component, e.g., a polyisocyanate. Generally, the first and the second packs are stored in separate containers and mixed before use. The containers are preferably sealed air tight to prevent degradation during storage. The mixing may be done, for example, in a mixing nozzle or in a container. When the crosslinking component contains, e.g., a polyisocyanate, the curing step can take place under ambient conditions, or if desired, it can take place at elevated baking temperatures.

For a two pack coating composition, the two packs are mixed just prior to use or 5 to 30 minutes before use to form a potmix. A layer of the potmix is typically applied to a substrate by the above conventional techniques. If used as a clear coating, a layer is applied over a metal substrate, such as, automotive body, which is often pre-coated with other coating layers, such as, an electrocoat primer, primer surfacer and a basecoat. The two-pack coating composition may be dried and cured at ambient temperatures or may be baked upon application for 10 to 60 minutes at baking temperatures ranging from 80° C. to 160° C. The mixture can also contain pigments and can be applied as a mono coat or a basecoat layer over a primed substrate.

The coating composition of the present invention is suitable for providing coatings on variety of substrates. Typical substrates for applying the coating composition of the present invention include automobile bodies, any and all items manufactured and painted by automobile sub-suppliers, frame rails, commercial trucks and truck bodies, including but not limited to beverage bottles, utility bodies, ready mix concrete delivery vehicle bodies, waste hauling vehicle bodies, and fire and emergency vehicle bodies, as well as any potential attachments or components to such truck bodies, buses, farm and construction equipment, truck caps and covers, commercial trailers, consumer trailers, recreational vehicles, including but not limited to, motor homes, campers, conversion vans, vans, pleasure vehicles, pleasure craft snow mobiles, all terrain vehicles, personal watercraft, motorcycles, bicycles, boats, and aircraft. The substrate further includes industrial and commercial new construction and maintenance thereof; cement and wood floors; walls of commercial and residential structures, such office buildings and homes; amusement park equipment; concrete surfaces, such as parking lots and drive ways; asphalt and concrete road surface, wood substrates, marine surfaces; outdoor structures, such as bridges, towers; coil coating; railroad cars; printed circuit boards; machinery; OEM tools; signage; fiberglass structures; sporting goods; golf balls; and sporting equipment.

The novel compositions of this invention are also suitable as clear or pigmented coatings in industrial and maintenance coating applications.

These and other features and advantages of the present invention will be more readily understood, by those of ordinary skill in the art from the following examples.

### Testing Procedures

The following test procedures were used for generating data reported in the examples below:

Chip Resistance Test

The test utilizes a gravelometer and follows the procedure described in ASTM-D-3170-87 using a 55° panel angle with panels and stones kept in the freezer for a minimum of 2 hours prior to chipping (panels were tested with 0.47 liter (1 pint)/1.42 liters (3 pints) of stones after a 20 minute @ 60° C. (140° F.) bake then air drying for an additional 7 days.

Gloss Measurement

Gloss was measured at 20° using a Byk-Gardener Glossmeter.

Distinctness of Image (DOI)

DOI was measured using a Dorigon II (HunterLab, Reston, Va.).

X-Hatch Adhesion and Grid Adhesion

X-Hatch Adhesion and Grid Adhesion were measured according to ASTM D 5339.

The invention is illustrated by the following Examples. All parts and percentages are on a weight basis unless otherwise noted.

### EXAMPLES

The following branched acrylic polymer solvent dispersion was prepared by first forming a macromonomer solution, polymerizing this solution with additional (meth)acrylate monomers to form a branched polymer solution and then removing solvent and adding non-solvent for the branched polymer to form a solvent responsive dispersion.

Preparation of Macromonomer Solution

To a twelve liter flask equipped with heating mantle, stirrer, condenser, nitrogen blanket, monomer and Initiator feed lines the following constituents are added: 1075.04 g of solvent (butyl acetate) 713.1 g of solvent (ethyl acetate) and a monomer mixture of the following (ethylhexyl methacrylate, isobornyl methacrylate, butyl methacrylate and hydroxyethyl methacrylate) consisting of 150.96 g ethylhexyl methacrylate (SIGMA), 75.48 g isobornyl methacrylate (ROHM & HASS), 226.44 g butyl methacrylate (SIGMA), and 50.32 g hydroxyethyl methacrylate (SIGMA). This mixture was then heated to reflux. To this flask was added at reflux a mixture of 47.54 g (ethyl acetate) 71.31 g (butyl acetate), 118.8 g (methyl ethyl ketone) 4.16 g 2,methylbutyronitrile (DUPONT) and 0.1188 g Bis (borondifluoro diphenylglyoximato) cobaltate (DUPONT) then held for five minutes. After the hold a monomer mixture of 1358.64 g ethylhexyl methacrylate, 679.79 isobornyl methacrylate, 2037.96 g, butyl methacrylate 452.88 g hydroxyethyl methacrylate and 48.96 g butyl acetate were fed over a period of 180 minutes. Simultaneously with the monomer feed, a mixture consisting of 427.86 g ethyl acetate, 641.79 g butyl acetate 37.44 g 2,methylbutyronitrile was added over a period of 300 minutes. Refluxing at a polymerization temperature of 90′C was maintained over the entire reaction time. After the monomer feed completion, 11 8.8 g of butyl acetate was used to rinse the monomer flask and added to the reaction flask, as was 42.9 g of butyl acetate used to rinse the initiator flask and added to the reaction flask. This mixture was held for a period of sixty minutes at reflux. After the hold, 118.8 g of ethyl acetate and 2.4 g of t-butyl peroxy 2-ethylhexanote was added as a shot, and held for thirty minutes at reflux. After the hold, the reaction flask was cooled to less than 70′c and the contents poured out.

The resulting macromonomer solution had a 69.79% solids content and a Gardner Holt Viscosity of V+½.

Preparation of a Branched Acrylic Polymer and Solvent Responsive Dispersion (SRD)

To a two-liter reaction flask equipped with heating mantle, condenser, stirrer, nitrogen blanket, monomer and initiator feed lines, the following were added: 150 g (ethyl acetate), 60 g (butyl acetate) and 304 g of above macromonomer solution and heated to reflux. To this flask a monomer mixture of (methyl methacrylate, hydroxy ethyl acrylate, acrylic acid, methyl acrylate and styrene) in a ratio of 35.01/10/4.99/35.01/14.99 consisting of 148.1 g methyl methacrylate (Cyro Industries ), 42.3 g hydroxyethyl acrylate (Dow Chemical) 21.1 g acrylic acid (Celanese Chemical) 148.1 g methyl acrylate(Celanese Chemical) 63.4G styrene (LANCASTER SYNTHESIS INC.) was added over a period of 60 minutes. Simultaneously with the monomer feed, a mixture consisting of 5 g of 2,4 dimethylvaleronitrile (DuPont Chemical) 155 g ethyl acetate and 50 g butyl acetate was added over a period of 360 minutes. Refluxing at a polymerization temperature of 90° C. was maintained over the entire reaction time. After the monomer feed was complete, 5 g of ethyl acetate was used to rinse the monomer flask and added to the reaction flask, as was 5 g of ethyl acetate was used to rinse the initiator flask, and added to the reaction flask. After all feeds and rinses were added, the mixture was then held 30 minutes at reflux temperature. The flask was further cooled to less than 70° C. and the contents poured out.

The branched acrylic polymer solution had a 51.44% solids content and the polymer had a GPC Mn of 24,231 and a Mw of 68,944. The Theoretical Tg of the polymer was 45° C.

To a 5 liter reaction flask equipped with heating mantle, stirrer, nitrogen blanket, condenser, water separator, addition funnel, the following were added: 2218 g of the branched acrylic polymer solution (prepared above) and heated to 70° C. To reduce solids to 40%, 634.0 g of heptane was added over 30 minutes. The mixture was further heated to a reflux temperature of 75° C. and 81 g of distillate was collected. The removed solvent was replaced with an equal amount of heptane, 81 g. The mixture was further cooled to less than 60° C. and the contents poured out. The resulting composition was a solvent responsive dispersion (SRD) having a 40.51% solids content and a Brookfleld Viscosity of 160 CPS @ 5 rpm. The theoretical Tg of the polymer was 45° C.

Preparation of Highly Branched Copolyester Polyol Solution

The following highly branched copolyester polyol solution was prepared and used to form coating composition:

A random highly branched copolyester polyol was synthesized by esterifying dimethylolpropionic acid, pentaerythritol and ε-caprolactone as follows:

The following constituents were charged into a 12-liter reactor equipped with a mechanical stirrer, thermocouple, short path distillation head with a water separator under nitrogen flow:

| | |
|---|---|
| Dimethylolpropionic acid (DMPA) | 1668.8 |
| Pentaerythritol (PE) | 67.6 |
| Tin(II)2-ethylhexanoate | 25.1 |
| ε-Caprolactone (CL) | 3337.6 |
| Xylene | 87.6 |

The reaction mixture was heated to its reflux temperature and the water of reaction was collected from the water separator. The reaction progress was monitored by the amount of water collected and the reaction temperature was not allowed to exceed 185° C. An additional 40 g of xylene was added throughout the reaction to maintain the reflux temperature below 185° C. When the amount of water collected approached theoretical amount of 224 g, acid number measurements were used to determine the end point, which was an acid number of less than 5. At a measured acid number of 3.0, the reactor was allowed to cool to 90° C. The reactor was held at 120° C. until reaction solids exceeded 95%. The reactor was allowed to cool to 90° C. and the polymer solution was thinned with 2537.3 g of polyethyleneglycol monomethyl ether. Forced air was used to cool the reactor to below 50° C.

The polymer had a Mn of 7065, Mw/Mn of 3.27 (determined by GPC using polystyrene as a standard with a SEC high MW column), an OH# equal to 166.8, and a calculated −OH EW of 335.8. The polymer solution has 65.6% solids content, a Gardner Holdt viscosity of V+½, and the final acid number of 2.5.

Basecoat Preparation

A Red Metallic Composite Tinting A was produced by mixing together, on an air mixer, the components shown below supplied by DuPont.

| Component | Description | Grams |
|---|---|---|
| 864J | DuPont MasterTint ® Magenta Tinting | 7884.55 |
| 813J | DuPont MasterTint ® Medium Coarse | 1010.06 |
| | Aluminum Tinting | |

| | |
|---|---|
| Total | 8894.61 |

A Solvent Blend B was prepared by mixing the following ingredients on an air mixer:

| Component | Grams |
|---|---|
| Butyl acetate | 7964.60 |
| Methyl amyl ketone | 3413.40 |
| Total | 11378.00 |

Basecoat lacquers of Comparative Example 1 and Examples 2 and 3 of the present invention were prepared by adding the components listed in Table 1 in order on an air mixer:

TABLE 1

| Component | Comp. Ex. 1 | Ex. 2 | Ex. 3 |
|---|---|---|---|
| Red Metallic Composite Tinting A | 518.42 | 518.42 | 518.42 |
| ChromaPremier ® 62320F Basecoat | 453.30 | — | — |
| Binder | | | |
| ChromaSystems ® 7175S Basemaker | 828.27 | — | — |
| SRD Dispersion (prepared above) | — | 96.34 | 62.62 |
| Highly Branched Copolyester Polyol- | — | — | 21.01 |
| Solution (prepared above) | | | |
| Solvent blend B (prepared above) | — | 929.00 | 942.00 |

### Panel Preparation

DuPont Variprime® Self-Etching Primer was prepared by mixing together 600 grams of 615S Variprime® with 400 grams of 616S Converter, all supplied by DuPont Company, Wilmington, Del. The Self-Etching Primer was sprayed according to the instructions in the ChromaSystem™ Technical Manual supplied by DuPont Company, Wilmington, Del. over cold rolled steel panels (sanded with Norton 80-D sandpaper supplied by Norton, Worcester, Mass., and wiped twice with DuPont 3900S First Klean™ supplied by DuPont Company, Wilmington, Del.) resulting in a film thickness of 25.4 to 28 micrometers (1.0 to 1.1 mils). The ChromaPremier® type basecoats (Samples 1 to 3) were then applied per the ChromaPremier® Basecoat instructions in the ChromaSystem™ Technical Manual, resulting in film thicknesses of 28 to 30 micrometers (1.1 to 1.2 mils). After flashing, 72200S ChromaPremier® Productive Clear (528 grams 72200S ChromaPremier® Productive Clear blended with 187 grams 12305S ChromaPremier® Activator and 185 grams 12375S ChromaPremier® Medium Reducer, all supplied by DuPont Company, Wilmington, Del.) was applied per the instructions in the ChromaSystem™ Technical Manual, resulting in a film thickness of about 56 micrometers (2.2 mils). After flashing, the panels were baked for 20 minutes at 60° C. (140° F.). The panels were then aged for one week at approximately 25° C. @ 50% relative humidity prior to testing.

### Test Results

Below in Table 2 are the gloss (using a BYK-Gardner glossmeter) and distinctness of image (using a Dorigon II meter) values:

TABLE 2

| | 20° Gloss | DOI |
|---|---|---|
| Basecoat | BC/CC | BC/CC |
| Comp. Ex. 1 | 86.8 | 89 |
| Ex. 2 | 85.7 | 89 |
| Ex. 3 | 85.9 | 85.9 |

This data shows that the use of solvent responsive dispersion in the lacquer basecoat did not adversely affect appearance.

The basecoat/clear coat panels were subjected to the chip resistance test described earlier. The results are shown in Table 3 below:

TABLE 3

Chip Resistance

| Basecoat* | 1 Pint | 3 Pints |
|---|---|---|
| Comp. Ex. 1 | 5 | 4.5 |
| Ex. 2 | 6 | 5 |
| Ex. 3 | 7 | 6 |

*All basecoats were further coated with the clear coat described above in panel preparation.

The data showed that the panels' chip performance particularly benefited from the use of solvent responsive dispersions in the lacquer basecoat.

Table 4 below shows the results of the X-hatch and grid hatch adhesion test (ASTM D3359) and DOI readings after 96 hours in the humidity cabinet (ASTM-D-2247-99) at 100% relative humidity. Readings were taken before exposure (initially), and after removal from the humidity cabinet (wet).

TABLE 4

| | X-Hatch Adhesion | | Grid Hatch Adhesion | | DOI |
|---|---|---|---|---|---|
| Basecoat* | Initial | Wet | Initial | Wet | Wet |
| Comp. Ex. 1 | 9.5 | 9 | 10 | 7 | 49.3 |
| Ex. 2 | 10 | 9 | 10 | 8 | 64 |
| Ex. 3 | 10 | 9.5 | 10 | 8 | 75.6 |

*All basecoats were further coated with the clear coat described above in panel preparation.

The data showed that the panels' moisture resistance benefited from the use of the solvent responsive dispersion in the basecoat.

What is claimed is:

1. A coating composition comprising 10% to 95% by weight, based on the weight of the coating composition of a liquid organic carrier, 5% to 90% by weight, based on the weight of the coating composition of a binder;
wherein the binder comprises a branched acrylic polymer having a glass transition temperature of −10° C. to 100° C. and a weight average molecular weight of 8000 to 150,000 comprising macromonomers formed from free radical polymerized ethylenically unsaturated monomers substantially having a terminal ethylenically unsaturated group polymerized with ethylenically unsaturated monomers thereby forming a branched acrylic polymer having a backbone of polymerized ethylenically unsaturated monomers and macromonomer branch chains;
wherein the ethylenically unsaturated monomers comprise a mixture of at least two different ethylenically unsaturated monomers wherein at least one of the monomers has the formula
CH₂═CXY
where X is H or CH₃ and
Y contains a group selected from the group of carboxyl, hydroxyl, primary amine, secondary amine, or tertiary amine.

2. The coating composition of claim 1 wherein the macromonomers are prepared by the free radical polymerization of ethylenically unsaturated monomers in the presence of a catalytic chain transfer agent containing Co⁺² or Co⁺³ thereby forming a single terminal ethylenically unsaturated group on each of the macromonomers.

3. The coating composition of claim 2 wherein the branched acrylic polymer comprises 30% to 70% by weight, based on the weight of the branched acrylic polymer, of macromonomer branch chains and 70% to 30% by weight, based on the weight of the branched acrylic polymer of a backbone of polymerized ethylenically unsaturated monomers.

4. The coating composition of claim 1 containing pigment in a pigment to binder ratio of 0.1/100 to 200/100.

5. The coating composition of claim 3 wherein the ethylenically unsaturated monomers are selected from the group consisting of hydroxy alkyl (meth)acrylates having 1-4 carbon atoms in the alkyl group, ethylenically unsaturated carboxylic acids, and any mixtures thereof, and the backbone and the macromonomers contain additional monomers selected from the group consisting of alkyl (meth)acrylates having 1-20 carbon atoms in the alkyl group, cycloaliphatic (meth)acrylates, styrene, alpha methyl styrene, vinyl toluene, acrylonitrile, methacrylonitrile, acrylamide, methacrylamide, glycidyl (meth)acrylate, isobornyl (meth)acrylate and any mixtures thereof.

6. The coating composition of claim 5 wherein the macromonomers consist essentially of polymerized monomers of ethyl hexyl methacrylate, isobornyl methacrylate, butyl methacrylate, hydroxy ethyl acrylate and the monomers of the backbone consist essentially of methyl methacrylate, hydroxy ethyl acrylate, acrylic acid, methyl acrylate and styrene.

7. The coating composition of claim 1 wherein the organic liquid carrier comprises a non-solvent for the binder and the composition comprises a fine dispersion of binder particles.

8. The coating composition of claim 1 containing in addition 5-50% by weight, based on the weight of the binder, of a highly branched polyester polyol.

9. The coating composition of claim 1 wherein the binder further comprises 0.1% to 20% by weight, based on the weight of the binder of cellulose acetate butyrate.

10. A process for forming a clear coat/base coat finish on a substrate which comprises applying the coating composition of claim 1 to the substrate and then applying a clear coating composition there-over and curing both layers to form the finish.

11. A substrate coated with a layer of the coating composition of claim 1.

12. The substrate of claim 11 top coated with a clear layer of a coating composition and fully cured to form a clear coat/base coat finish on the substrate.

13. The coating composition of claim 1 wherein the binder contains 0.1-50% by weight, based on the weight of the binder of an organic polyisocyanate crosslinking agent.

14. The coating composition of claim 4 wherein the pigment comprises flake pigments.

15. The coating composition of claim 1 wherein the organic carrier is an organic solvent and wherein the composition is a lacquer composition that dries via solvent evaporation and forms a substantially un-crosslinked coating.

16. The coating composition of claim 15 containing in addition a polyester, an alkyd resin, an acrylic alkyd resin, cellulose acetate butyrate, an iminated acrylic polymer, an ethylene vinyl acetate copolymer, nitrocellulose, plasticizer and any combinations thereof.

17. The coating composition of claim 4 wherein the organic carrier is an organic solvent and wherein the composition is a lacquer composition that dries via solvent evaporation and forms a substantially un-crosslinked coating.

18. The coating composition of claim 17 containing in addition a polyester, an alkyd resin, an acrylic alkyd resin, cellulose acetate butyrate, an iminated acrylic polymer, an ethylene vinyl acetate copolymer, nitrocellulose, plasticizer and any combinations thereof.

19. A substrate coated with the coating composition of claim 17 and having a clear coating composition applied thereover.
